(19) United States
(12) Patent Application Publication
(10) Pub. No.: US 2026/0151118 A1
(43) Pub. Date: Jun. 4, 2026

(54) SHEATH FOR ENABLING NEEDLE EXCHANGE AND NEEDLE-SHARP SAFETY

(71) Applicant: Boston Scientific Scimed, Inc., Maple Grove, MN (US)

(72) Inventors: Austin Grant Johnson, Worcester, MA (US); Seamus Finbarr O'Shaughnessy, Brighton, MA (US); Jeremy DiTullio, North Grafton, MA (US); Colby Harris, Norfolk, MA (US); Kevin Walsh, Marlborough, MA (US); Molly Phillips-Hungerford, Somerville, MA (US); Nancy Lieberman, Boston, MA (US)

(73) Assignee: Boston Scientific Scimed, Inc., Maple Grove, MN (US)

(21) Appl. No.: 19/353,393

(22) Filed: Oct. 8, 2025

Related U.S. Application Data
(63) Continuation of application No. 16/446,309, filed on Jun. 19, 2019, now abandoned.
(60) Provisional application No. 62/687,676, filed on Jun. 20, 2018.

(51) Int. Cl. A61B 10/02; A61B 10/04; A61B 17/34; A61B 90/00; A61M 25/00; A61M 25/06

(52) U.S. Cl. CPC A61B 10/0233; A61B 10/04; A61B 17/3403; A61M 25/0618; A61B 2010/045; A61B 2017/3413; A61B 2090/0801; A61M 25/0082

(57) ABSTRACT

The present disclosure relates generally to the field of medical devices. In particular, the present disclosure relates to medical systems and methods configured to shield the sharpened distal end of needles, such as exchangeable biopsy needles. The biopsy needles may be used for tissue biopsy and sampling under radial ultrasound guidance.

### CROSS REFERENCE TO RELATED APPLICATIONS

This application is continuation of U.S. Non-Provisional application Ser. No. 16/446,309, filed Jun. 19, 2019, which claims the benefit of priority under 35 U.S.C. § 119 to U.S. Provisional Patent Application Ser. No. 62/687,676, filed on Jun. 20, 2018, which is incorporated by reference in its entirety for all purposes.

### FIELD

The present disclosure relates generally to the field of medical devices. In particular, the present disclosure relates to medical systems and methods configured to shield the sharpened distal end of needles, such as exchangeable biopsy needles. The biopsy needles may be used for tissue biopsy and sampling under radial ultrasound guidance.

### BACKGROUND

As an example of systems utilizing needles, endoscopic ultrasound biopsy systems with exchangeable biopsy needles allow biopsy samples to be obtained from potentially cancerous pulmonary nodules under radial ultrasound guidance. These systems may include exchangeable needles to eliminate the need for the entire system to be removed from the patient, e.g., through the endoscope working channel, after each biopsy sample is taken. Exchangeable needles may improve procedure time by allowing a second biopsy sample to be taken after the previous sample is expelled for analysis, and may improve efficiency by maintaining the ultrasound image of the target nodule throughout the procedure. Once the needle is removed from the biopsy system, the exposed needle tip may pose a puncture/needle-stick risk to medical professionals in the operating room and/or technicians in the diagnostic laboratory. In addition, the tortuous anatomies within which exchangeable needle systems are deployed may result in puncture/skiving of the delivery catheter wall by the sharpened end of the biopsy needle. This skiving may result in a partial or incomplete biopsy sample and/or prevent the biopsy needle from completely retracting into the delivery catheter, thereby further promoting the potential for a puncture/needle stick.

A variety of advantageous medical outcomes may therefore be realized by the systems and/or methods of the present disclosure, which provide the combined benefits of a biopsy needle assembly that may be exchangeable, may prevent or eliminate puncture or skiving of the delivery catheter during a biopsy procedure, and may shield the exposed sharpened needle tip while still allowing the biopsy sample to be expelled for analysis.

### SUMMARY

In one aspect, the present disclosure relates to a needle assembly comprising a sheath which may include a proximal end, a distal end and a lumen extending therebetween. A needle comprising a proximal end and a sharpened distal end may be disposed within the lumen of the sheath. An actuation mechanism may be disposed along a proximal portion of the needle assembly. The actuation mechanism may include a housing. A channel may be formed within a distal portion of the housing. A spring may be disposed within a chamber of the housing. A push button may be engaged with a proximal portion of the housing. The push button may be configured to move from a first position to a second position within the housing. A proximal portion of the sheath may be immovably disposed within the channel of the housing. A proximal portion of the needle may extend through the spring, e.g., such that the proximal end of the needle may contact an inner surface of the push button. The needle may move in a distal direction relative to the sheath when the push button moves distally within the chamber, and the needle may move in a proximal direction relative to the sheath when the push button moves proximally within the chamber. The proximal end of the housing may include a surface feature configured to engage a corresponding surface feature on a distal end of the push button. The push button may be configured to move between a first position and a second position within the chamber of the housing. The sharpened distal end of the needle may be positioned proximal to the distal end of the sheath when the push button is in the first position, and the sharpened distal end of the needle may be positioned distal to the distal end of the sheath when the push button is in the second position. An inner surface, or outer surface, or both inner and outer surface, of the sheath may include a low friction material.

In another aspect, the present disclosure relates to a needle assembly comprising a sheath which may include a proximal end, a distal end and a lumen extending therebetween. A needle comprising a proximal end and a sharpened distal end may be disposed within the lumen of the sheath. An actuation mechanism may be disposed along a proximal portion of the needle assembly. The actuation mechanism may include a housing. A channel may extend through a portion of the housing. A slot may extend through a wall of the housing. An arm may extend through the slot. A proximal portion of the sheath may be slidably disposed within a distal portion of the channel. A proximal portion of the needle may extend through sheath and through a length of the channel, e.g., to contact an inner surface of the actuation mechanism. A first end of the arm may be attached to the proximal end of the sheath. A second end of the arm may extend outside of the actuation mechanism. The sheath may move in a proximal direction relative to the needle when the arms moves proximally along the slot, and the sheath may move in a distal direction relative to the needle when the arm moves distally along the slot. The arm may be configured to move between a first position and a second position along the slot. The sharpened distal end of the needle may be positioned proximal to the distal end of the sheath when the arm is in the first position, and the sharpened distal end of the needle may be positioned distal to the distal end of the sheath when the arm is in the second position.

In another aspect, the present disclosure relates to a system comprising a catheter which may include proximal end, a distal end, a first lumen extending therebetween, and a second lumen extending therebetween. A needle assembly may be disposable within the first lumen. A radial ultrasound probe may be disposed within the second lumen. The needle assembly may include a needle disposed within a sheath. The needle assembly may be movable from a first position in which a sharpened distal end of the needle is positioned proximal to a distal end of the sheath and a second position in which the sharpened distal end of the needle is positioned distal to the distal end of the sheath. An actuation mechanism may be disposed along a distal portion of the needle assembly. The actuation mechanism may include a housing. A channel may be formed within a distal portion of the housing. A spring may be disposed within a chamber of the housing. A push button may be engaged with a proximal portion of the housing. The push button may be configured to move within the housing. A proximal portion of the sheath may be immovably disposed within the channel of the housing. A proximal portion of the needle may extend through the spring, e.g., such that the proximal end of the needle may contact an inner surface of the push button. The needle may move in a distal direction relative to the sheath when the push button moves distally within the chamber, and the needle may move in a proximal direction relative to the sheath when the push button moves proximally within the chamber. The proximal end of the housing may include a surface feature configured to engage a corresponding surface feature on a distal end of the push button. The push button may be configured to move between a first position and a second position within the chamber of the housing. An inner surface, or outer surface, or both inner and outer surface, of the sheath may include a low friction material. An inner surface of the catheter may include a low friction material. The needle assembly may include an actuation mechanism disposed along a proximal portion of the needle assembly. The actuation mechanism may include a housing. A channel may extend through a portion of the housing. A slot may extend through a wall of the housing. An arm may extend through the slot. A proximal portion of the sheath may be slidably disposed within a distal portion of the channel. A proximal portion of the needle may extend through the sheath and through a length of the channel, e.g., to contact an inner surface of the actuation mechanism. A first end of the arm may be attached to the proximal end of the sheath. A second end of the arm may extend outside of the actuation mechanism. The sheath may move in a proximal direction relative to the needle when the arms moves proximally along the slot, and the sheath may move in a distal direction relative to the needle when the arm moves distally along the slot. The arm may be configured to move between a first position and a second position along the slot. The sharpened distal end of the needle may be positioned proximal to the distal end of the sheath when the arm is in the first position, and the sharpened distal end may be positioned distal to the distal end of the sheath when the arm is in the second position. An inner surface, or outer surface, or both inner and outer surface, of the sheath may include a low friction material. An inner surface of the catheter may include a low friction material. The sharpened distal end of the needle may include a three-point needle grind. At least a portion of the sheath may include an optically translucent material. One or more grooves may be formed along a length of the sheath. A cross-section of the sheath may include a circular, elliptical, oval or star-shaped geometry.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1D provide perspective views of a needle assembly, according to one embodiment of the present disclosure.

FIGS. 2A-2B provide perspective views of a needle assembly, according to one embodiment of the present disclosure.

FIGS. 3A-3D provide perspective views of a needle assembly and catheter, according to one embodiment of the present disclosure.

FIG. 4 provides a front-perspective view of a catheter, according to one embodiment of the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical systems and methods for acquiring biopsy samples from a pulmonary nodule under radial ultrasound guidance, it should be appreciated that such systems and methods may be used in a variety of medical procedures where there is a need for exchangeable or single-use needle assemblies that shield the exposed needle tip while still allowing the needle tip to be accessible, including, for example, in endoscopy procedures, intravenous procedures, etc.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

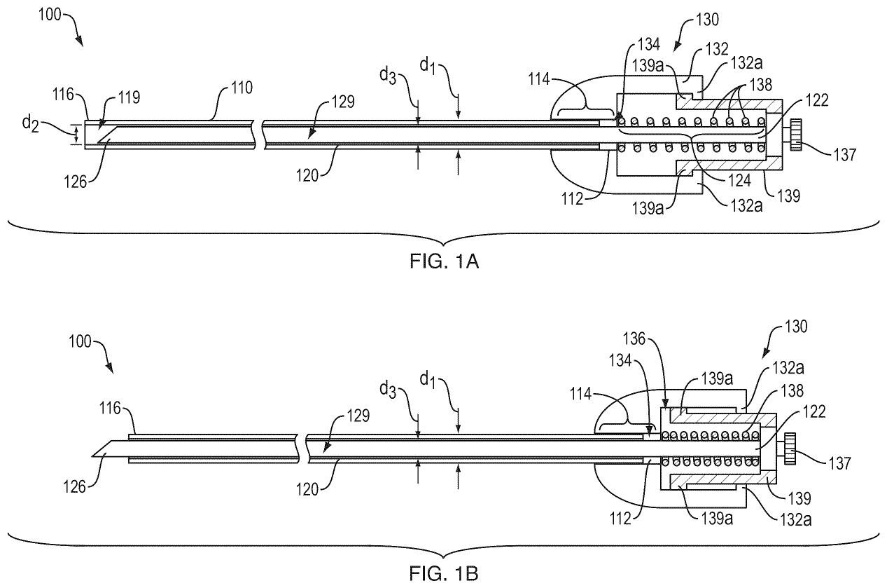

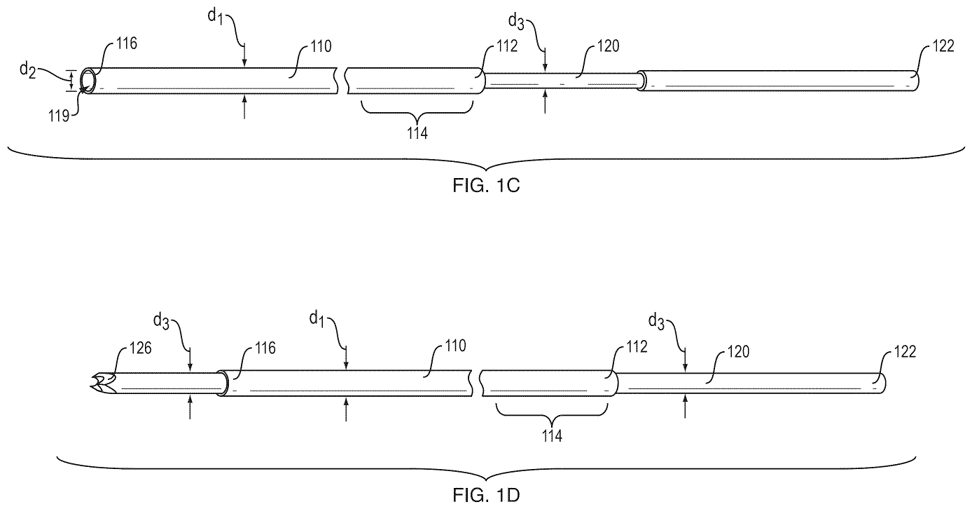

In various embodiments, the present disclosure relates to devices and methods to shield the exposed tip of an exchangeable biopsy needle following removal from the patient. Referring to FIGS. 1A-1D, in one embodiment, a needle assembly 100 (e.g., biopsy needle assembly) of the present disclosure may include a biopsy needle 120 slidably disposed within a sheath 110. The sheath 110 may include a proximal end 112, a distal end 116 and a lumen 119 extending therebetween. In various embodiments, the sheath 110 may include an outer dimension d₁ and an inner dimension d₂ (e.g., of lumen 119). The needle 120 may include proximal end 122, a sharpened distal end 126 and a lumen 129 extending therebetween. In various embodiments, the biopsy needle 120 may include an outer dimension d₃ less than the inner dimension d₂ of the lumen 119 of sheath 110.

An actuation mechanism 130 may be disposed along a proximal portion of the needle assembly 100. The actuation mechanism 130 may include a housing 132, comprising a channel 134 formed within a distal portion of the housing 132 (e.g., along a longitudinal axis of the housing) and a chamber 136 formed within a proximal portion of the housing 132. A spring 138 may be disposed within, and extend along a longitudinal length of, the chamber 136. A push button 139 may be engaged with the proximal portion of the housing 132 and configured to move between a plurality of distally advanced and proximally retracted positions within the chamber 136. In various embodiments, the proximal portion of the housing 132 may include one or more surface features 132a configured to engage one or more corresponding surface features 139a formed along the distal portion of the push button 139. The surface features 132a, 139a may be configured to contact each other to maintain the push button 139 within the housing 132 (e.g., prevent separation) when the push button 139 is in the proximally retracted configuration. In various embodiments, the surface features 132a, 139a may include one or more inward and outward extending tabs (respectively) disposed in various patterns (including a fully circumferential patterns) along the respective proximal and distal ends of the housing 132 and push button 139. In addition, or alternatively, the housing 132 and push button 139 may include a variety of configurations to prevent unintended separation, including, for example, corresponding thread or luer-lock connector configurations, and the like.

A proximal portion 114 of the sheath 110 may be immovably attached or affixed to the distal portion of the housing 132. In various embodiments, the proximal portion 114 of the sheath 110 may be bonded, adhered and/or integrally formed with at least a portion of the channel 134 of housing 132. A proximal portion 124 of the needle 120 may extend proximally beyond the proximal end 112 of the sheath 110, and through the spring 138 within the chamber 136, such that the proximal end 122 of the needle 120 contacts (or is adjacent to) an inner surface of the push button 139. In various embodiments, the proximal end 122 of the needle 120 may be attached to a stylet 137 extending through an opening (not shown) formed within a proximal end of the push button 139. The stylet 137 may extend through all, or a portion, of the lumen 129 of the needle 120. The stylet may obstruct or occlude the lumen 129, e.g., as the needle is advanced through narrow and tortuous body passages. The stylet 137 may be removed to provide access to the lumen 129, e.g., to expel or aspirate a biopsy sample, or to inject a fluid into or around the target pulmonary nodule.

In various embodiments, the push button 139 may be distally advanced/depressed, e.g., under the force applied by a thumb and forefinger of a user, to compress the spring 138 within the chamber 136, thereby distally advancing the needle 120 relative to the sheath 110 such that the sharpened distal end 126 of the needle 120 moves from a first position (e.g., shielded, retracted or protected position) within the lumen 119 proximal to the distal end 116 the sheath 110 (FIG. 1A), to a second position (e.g., exposed, extended, unprotected) distally beyond (e.g., positioned distal to) the distal end 116 of the sheath 110 (FIG. 1B). Similarly, the push button 139 may be proximally retracted/released, e.g., by removing or reducing the distal force exerted by the user, such that the spring 138 returns to the relaxed (e.g., non-compressed) position, thereby retracting the sharpened distal end 126 of the needle 120 into the lumen 119 of the sheath 110.

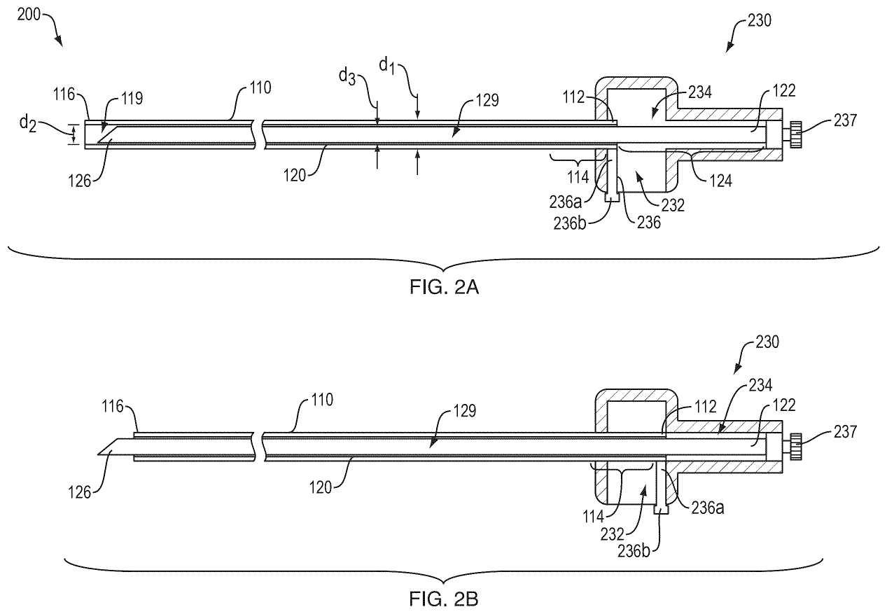

Referring to FIGS. 2A and 2B, in one embodiment, a needle assembly 200 (e.g., biopsy needle assembly) of the present disclosure may include a sheath 110 and needle 120, e.g., as depicted in FIGS. 1C and 1D, with the exception that the sheath 110 is slidably disposed around at least a portion of the needle 120. The sheath 110 may include a proximal end 112, a distal end 116 and a lumen 119 extending therebetween. The sheath 110 may include an outer dimension d₁ and an inner dimension d₂ (e.g., of lumen 119). The needle 120 may include a proximal end 122, a sharpened distal end 126 and a lumen 129 extending therebetween. The biopsy needle may include an outer dimension d₃ less than the inner dimension d₂ of the lumen 119 of sheath 110.

An actuation mechanism 230 may be disposed along a proximal portion of the needle assembly 200. The actuation mechanism 230 may include a housing 232, a channel 234 may extend through/along a length (e.g., longitudinal axis) of the housing 232 and a slot or opening 233 may extend through a wall of the housing 232 in communication with (e.g., opening into) a distal portion of the channel 234. An arm 236 (e.g., slider arm, trigger, finger grip, etc.) may extend through the slot 233 of the housing 232 such that a first end 236a of the arm 236 extends into, or is substantially coextensive with, the channel 234 and a second end 236b of the arm extends outside the housing 232.

A proximal portion 114 of the sheath 110 may be slidably/movably disposed within the distal portion of the channel 234. The first end 236a of the arm 236 may be attached to, or integrally formed with, the distal end 116 of the sheath 110. A proximal portion 124 of the needle 120 may extend proximally beyond the proximal end 112 of the sheath 110 and through the remaining (e.g., proximal portion) of the channel 234 such that the proximal end 122 of the needle 120 contacts (or is adjacent to) an inner surface of the housing 232. In various embodiments, the proximal end 122 of the needle 120 may be attached to a stylet 237 extending through an opening (not shown) formed within a proximal end of the housing 232. As above, the stylet 237 may extend through all, or a portion, of the lumen 129 of the needle 120. The stylet may obstruct or occlude the lumen 129, e.g., as the needle is advanced through narrow and tortuous body passages. The stylet 137 may be removed to provide access to the lumen 129, e.g., to expel or aspirate a biopsy sample, or to inject a fluid into or around the target pulmonary nodule.

In various embodiments, the arm 236 may be proximally retracted along the slot 233, e.g., under the force applied by a thumb and forefinger of a user, thereby proximally retracting the sheath 110 relative to the needle 120 such that the sharpened distal end 126 of the needle 120 moves from a first position (e.g., shielded, retracted or protected position) within the lumen 119 proximal to the distal end 116 the sheath 110 (FIG. 2A), to a second position (e.g., exposed, extended, unprotected) distally beyond the distal end 116 of the sheath 110 (FIG. 2B). Similarly, the arm 236 may be distally advanced along the slot 233 such that the sheath 110 moves along the needle 120 to position/reposition the distal end 116 of the sheath 110 distally beyond the sharpened distal end 126 of the needle 120.

In various embodiments, a spring (not shown) may be disposed within a portion of the channel 234 proximally beyond the proximal end of the sheath 110, and with the proximal portion of the needle 120 extending through the spring, as discussed above. As the arm 236 is proximally retracted along the slot 233, the spring may compress within the channel 234 to expose the sharpened distal end 126 of the needle 120. The arm 236 and sheath 110 may then return to the first position by partially or fully releasing the pressure exerted on the arm 236 such that the spring 138 returns to the relaxed (e.g., non-compressed) position.

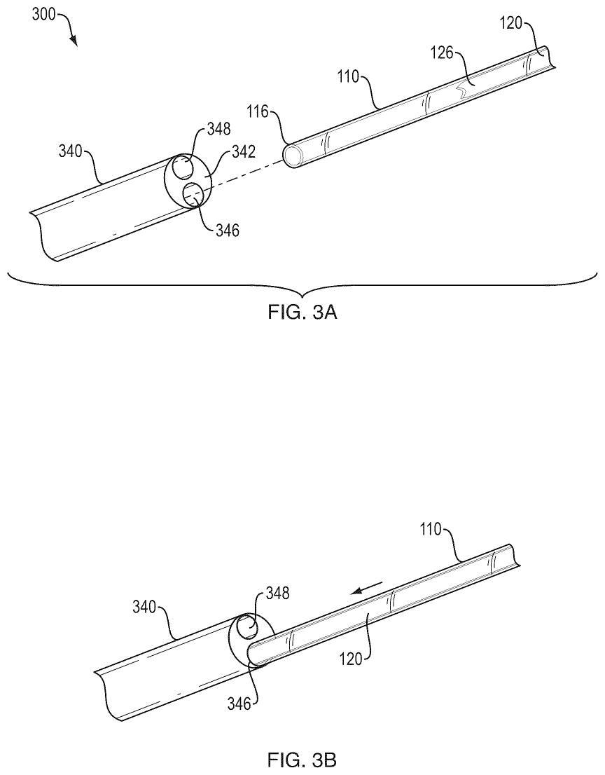

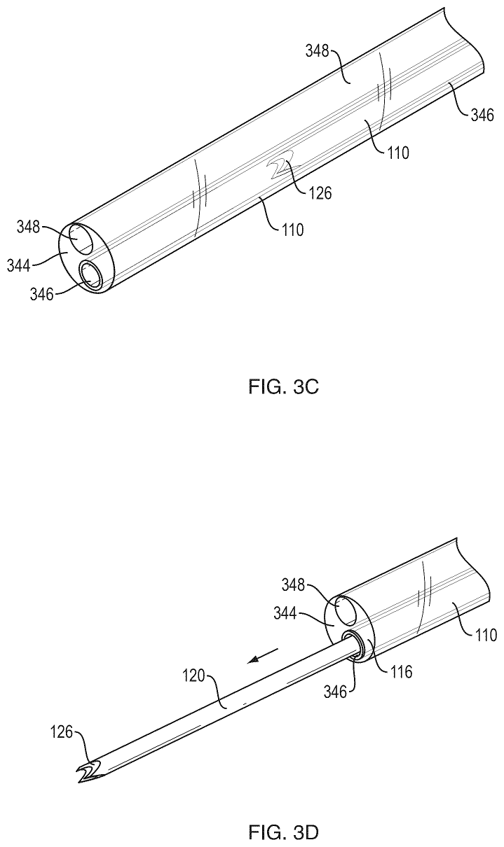

Referring to FIGS. 3A-3D, in one embodiment, a system 300 of the present disclosure may include a needle assembly 100, 200 slidably disposed within a dual-lumen catheter 340. In various embodiments, the dual-lumen catheter 340 may include a proximal end 342, a distal end 344, a first lumen 346 and a second lumen 348 extending therebetween. The first lumen 346 may include an inner dimension d₄ sized and configured to receive the outer dimension d₁ of the sheath 110 of the needle assembly 100, 200. The second lumen 348 may include an inner dimension d₅ sized and configured to receive a radial ultrasound probe (not shown).

Referring to FIG. 3C, the sheath 110 and needle 120 of the needle assembly 100, 200 may be advanced in the first position (FIGS. 1A, 2A) through the first lumen 346 to position the distal end 116 of the sheath 110 adjacent to (or substantially coextensive with) the distal end 344 of the dual-lumen catheter 340.

Referring to FIG. 3D, in one embodiment, the needle assembly 100 may be moved from the first to second position, e.g., by distally advancing push button 139 (as discussed above) to advance the sharpened distal end 126 of the needle 120 distally beyond the respective distal ends 116, 344 of the sheath 110 and dual-lumen catheter 340. Still referring to FIG. 3D, in another embodiment, the needle assembly 200 may be moved from the first to second position, e.g., by proximally retracting the arm 236 and sheath 110 (as discussed above), to such that the sharpened distal end 126 of the needle 120 extends distally beyond the distal end 116 of the sheath 110 within the first lumen 346 of the dual-lumen catheter 340. The needle assembly 200, including the exposed sharpened distal end 126 of the needle 120 may then be further distally advanced through the first lumen 346 while maintaining the arm 236 and sheath 110 in the second position, such that the sharpened distal end 126 of the needle 120 distally beyond the distal end 344 of the dual-lumen catheter 340.

In use, and by way of example, a needle biopsy system 300 of the present disclosure may be inserted into a pulmonary passage of a patient through a working channel of an endoscope and adjacent to a target pulmonary nodule. The stylet 137 may then be removed from the lumen of the biopsy needle 120, and the sharpened distal end 126 of the needle 120 advanced distally beyond the distal end 344 of the dual-lumen catheter 340 into the target pulmonary nodule (as discussed above). The user may distally deploy the needle 120 into the target pulmonary nodule as many times as necessary to acquire sufficient tissue for analysis. When the user determines that the biopsy needle contains a sufficient amount of tissue from the target pulmonary nodule, the needle assembly 100, 200 may be returned to the first position (e.g., with the sharpened distal end 126 positioned proximal to the distal end 116 of the sheath 110), and the needle assembly 100, 200 proximally withdrawn from the first lumen 346 of the dual-lumen catheter 340. The sharpened distal end 126 of the needle is therefore shielded from the user and/or ancillary personnel immediately upon removal from the sheath 110. In various embodiments, the needle assembly 100, 200 may be removed from within the first lumen 346 of the dual-lumen catheter 340 without changing the position of the dual-lumen catheter 340 and radial ultrasound probe relative to the target pulmonary nodule.

In various embodiments, the tissue sample may then be expelled from the needle assembly while the sharpened distal end 126 remains safely housed within the sheath 110. Alternatively, the sharpened distal end 126 of the needle 120 may be advanced distally beyond the distal end 116 of the sheath 110 prior to expelling the tissue sample. The needle assembly 100, 200 may then be proximally retracted into the sheath 110, and the needle assembly 100, 200 re-introduced through the first lumen 346 of the dual-lumen catheter 340 to acquire an additional biopsy sample from the same, or a different, pulmonary nodule. Alternatively, one or more different needle assemblies 100, 200 may be introduced through the first lumen 346 of the dual-lumen catheter 340 while the biopsy sample is being expelled from the original needle assembly 100, 200. The ability to obtain multiple additional biopsy samples from the same or different pulmonary nodule while the biopsy sample is expelled from the original (e.g., first needle assembly) may further improve procedure time, efficiency and accurate/reliable diagnostic results.

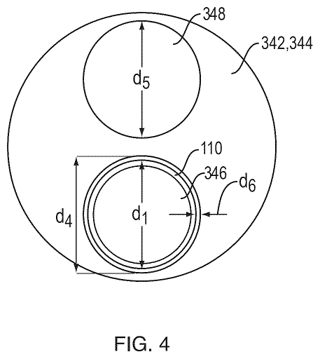

In various embodiments, the respective inner and outer dimensions (d₁-d₄) of the sheath 110, needle 120 and first lumen 346 may be configured to minimize or prevent puncture or skiving of the sheath 110 and/or dual-lumen catheter 340 by the sharpened distal end 126 of the needle 120, e.g., within narrow and tortuous body passages in which the needle biopsy system 300 may be bent or torqued at a variety of angles during biopsy sample collection. Referring to FIG. 4, in one embodiment, the inner dimension d₄ of the first lumen 346 and the outer dimension d₁ of the sheath 110 may be separated by a distance d₆ (e.g., approximately 0.0002 inch) configured to allow the needle assembly 100, 200 to move smoothly (e.g., with low or minimal friction) within the first lumen 346 with minimal lateral movement of the needle assembly 100, 200, e.g., which might contribute to puncture or skiving of the sheath 110 and/or dual-lumen catheter 340 during biopsy sample collection or expulsion.

In various embodiments, the sheath 110 may comprise a variety of low friction materials along all or a portion of the length of the sheath to minimize friction between the first lumen 346 of the dual-lumen catheter 340 and/or biopsy needle 120. The sheath 110 may be formed entirely from these low friction materials, or provided as inner and/or outer coatings of the sheath. Non-limiting examples of low-friction materials includes, Pebax, loaded Pebax, BaSO₄, PTFE, Mediglide™, Polymide, PEEK and glass-fiber reinforced PEEK. In addition, or alternatively, the first and/or second lumens 346, 348 of the dual-lumen catheter 340 may include a coating or liner of any of these low friction materials. In addition, or alternatively, at least a portion of the sheath 110 may comprise an optically translucent material (e.g., glass, plastic, rubber, etc.) to allow a user to visualize the sharpened end the needle 120 and/or biopsy sample disposed therein.

In various embodiments, the sharpened distal end 126 of the needle 120 may include a needle grind configured to further reduce the potential for puncture or skiving of the sheath 110 and/or dual-lumen catheter 340. For example, the needle 120 may include a three-point “Francine” needle grind, in which the sharpened points of the needle 120 extend slightly inward toward the lumen of the needle to further minimize the potential for puncture or skiving of the sheath or dual-lumen catheter. In addition to reducing friction, these low friction materials and/or coatings may further reduce the likelihood or ability of the sharpened distal end of the needle to puncture or skive the sheath and/or dual-lumen catheter. To further reduce the puncture or skiving of the sheath and/or dual-lumen catheter, in various embodiments, the needle 120 may be formed from or otherwise include a variety of flexible and/or kink-resistant materials (e.g., Nitnol, etc.) along all, or a portion of, the length of the needle 120.

In various embodiments, the sheath 110 may be formed from or include a variety of metallic, plastic, fiber or composite materials, or combinations thereof, along all or a portion of the length of the sheath. Such materials may provide the requisite mechanical strength for insertion of the sheath 110 into, and removal from, the tortuous path of the dual-lumen catheter 340 within the body, and/or minimize the potential for skiving by the sharpened distal end 126 of the needle 120. As will be understood by those of skill in the art, such metallic, plastic, fiber and/or composite materials may also reduce or eliminate the tendency of the sheath 110 to compress and/or stretch during insertion into, and removal from the dual-lumen catheter 340.

In addition, or alternatively, one or more slots or grooves may be included (e.g., laser slotted) along all or various portions of the length of the sheath 110 and/or dual-lumen catheter 340 to further increase the flexibility of the needle biopsy system 300, e.g., within tortuous body passages. In various embodiments, the sheath 110 is not limited to unitary or solid configurations, but may be formed from a hypotube, coil and/or braid along all or a portion of the length of the sheath.

In various embodiments, the sheath 110 and first lumen 346 of the dual-lumen catheter 340 are not limited to circular configurations, but may include a variety of cross-sectional shapes and/or geometries, including, for example, elliptical, oval or star-shaped geometries. In various embodiments, these geometries may minimize the surface area of the sheath 110 in contact with the inner wall of first lumen 346 the dual-lumen catheter 340, thereby further reducing friction and the potential for puncture or skiving by the sharpened distal end 126 of the needle 120.

The needle assemblies 100, 200 of the present disclosure are in no way limited to systems or methods that include dual-lumen catheters, but may be employed in a variety of single or multi-lumen catheters, including by way of non-limiting example, triple-lumen catheters, quadruple-lumen catheters and the like. In addition, the systems or methods of the present disclosure are in no way limited to use with radial ultrasound probes, but may include a variety of imaging modalities, including, for example, linear ultrasound probes, etc. Alternatively, the systems or method of the present disclosure may be used in standalone procedures that do not include any form of imaging modality or probe.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A system, comprising:
a catheter, comprising:
a proximal end,
a distal end,
a first lumen extending between the proximal end and the distal end, and
a second lumen extending between the proximal end and the distal end;
a needle assembly disposed within the first lumen; and
a radial ultrasound probe disposed within the second lumen;
wherein the needle assembly includes a needle disposed within a sheath, and
wherein the needle assembly is movable from a first position in which a sharpened distal end of the needle is positioned proximal to a distal end of the sheath and a second position in which the sharpened distal end of the needle is positioned distal to the distal end of the sheath.

2. The system of claim 1, wherein the needle assembly includes an actuation mechanism disposed along a proximal portion of the needle assembly, the actuation mechanism comprising:
a housing;
a channel formed within a distal portion of the housing;
a spring disposed within a chamber of the housing; and
a push button engaged with a proximal portion of the housing, the push button configured to move within the housing,
wherein a proximal portion of the sheath is immovably disposed within the channel of the housing,
wherein a proximal portion of the needle extends through the spring, and
wherein the needle moves in a distal direction relative to the sheath when the push button moves distally within the chamber and the needle moves in a proximal direction relative to the sheath when the push button moves proximally within the chamber.

3. The system of claim 2, wherein the proximal end of the housing includes a surface feature configured to engage a corresponding surface feature on a distal end of the push button.

4. The system of claim 3, wherein the push button is configured to move between a first position and a second position within the chamber of the housing.

5. The system of claim 1, wherein an inner surface, or outer surface, or both the inner and outer surface, of the sheath includes a low friction material.

6. The system of claim 1, wherein an inner surface of the catheter includes a low friction material.

7. The system of claim 1, wherein the needle assembly includes an actuation mechanism disposed along a proximal portion of the needle assembly, the actuation mechanism comprising:
a housing;
a channel extending through a portion of the housing;
a slot extending through a wall of the housing; and
an arm extending through the slot,
wherein a proximal portion of the sheath is slidably disposed within a distal portion of the channel,
wherein a proximal portion of the needle extends through the sheath and through a length of the channel,
wherein a first end of the arm is attached to the proximal end of the sheath, and a second end of the arm extends outside of the actuation mechanism, and
wherein the sheath moves in a proximal direction relative to the needle when the arm moves proximally along the slot, and the sheath moves in a distal direction relative to the needle when the arm moves distally along the slot.

8. A needle assembly, comprising:
a sheath comprising a proximal end, a distal end and a lumen extending therebetween;
a needle disposed within the lumen of the sheath, the needle comprising a proximal end and a sharpened distal end; and
an actuation mechanism disposed along a proximal portion of the needle assembly, the actuation mechanism comprising a housing, a channel formed within a distal portion of the housing, a spring disposed within a chamber of the housing and a push button engaged with a proximal portion of the housing, the push button configured to move from a first position to a second position within the housing,
wherein a proximal portion of the sheath is immovably disposed within the channel of the housing,
wherein a proximal portion of the needle extends through the spring,
wherein the needle moves in a distal direction relative to the sheath when the push button moves distally within the chamber and the needle moves in a proximal direction relative to the sheath when the push button moves proximally within the chamber.

9. The needle assembly of claim 8, wherein the proximal end of the housing includes a surface feature configured to engage a corresponding surface feature on a distal end of the push button.

10. The needle assembly of claim 8, wherein an inner surface, or outer surface, or both inner and outer surface, of the sheath includes a low friction material.

11. A system, comprising:
a catheter, comprising:
a proximal end,
a distal end,
a first lumen extending therebetween, and
a second lumen extending therebetween;
a needle assembly disposed within the first lumen; and
a radial ultrasound probe disposable within the second lumen;
wherein the needle assembly includes a needle disposed within a sheath,
wherein the needle assembly is movable from a first position in which a sharpened distal end of the needle is positioned proximal to a distal end of the sheath and a second position in which the sharpened distal end of the needle is positioned distal to the distal end of the sheath, and
wherein the needle assembly includes an actuation mechanism disposed along a proximal portion of the needle assembly, the actuation mechanism comprising:
a housing,
a channel formed within a distal portion of the housing,
a spring disposed within a chamber of the housing, and
a push button disposed at least partially within the chamber of the housing proximal to the spring, the push button configured to repeatedly move along a longitudinal axis of the housing;
wherein a proximal portion of the sheath is immovably disposed within the channel of the housing;
wherein a proximal portion of the needle extends through the spring and is configured to contact an inner surface of the push button such that when the push button moves between the first position to the second position the needle moves in a distal direction relative to the sheath and when the push button moves from the second position to the first position the needle moves in a proximal direction relative to the sheath;
wherein when in the first position the sharpened distal end of the needle is disposed within the sheath.

12. The system of claim 11, wherein the proximal portion of the housing includes a surface feature configured to engage a corresponding surface feature on a distal end of the push button.

13. The system of claim 11, wherein the push button is configured to move between the first position and the second position within the chamber of the housing.

14. The system of claim 11, wherein an inner surface, or outer surface, or both inner and outer surface, of the sheath includes a low friction material.

15. The system of claim 11, wherein an inner surface of the catheter includes a low friction material.

16. The system of claim 11, wherein the sharpened distal end of the needle includes a three-point needle grind.

17. The system of claim 11, wherein the push button is biased toward the first position by the spring.
